(19) United States
(12) Patent Application Publication
(10) Pub. No.: US 2004/0058000 A1
(43) Pub. Date: Mar. 25, 2004

(54) Controlled release capsule for delivery of liquid formulation

(76) Inventors: Liang C. Dong, Sunnyvale, CA (US); Keru Shafi, Anaheim Hills, CA (US); Alicia Yum, Belmont, CA (US); Patrick S. L. Wong, Burlingame, CA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/608,305

(22) Filed: Jun. 27, 2003

Related U.S. Application Data
(60) Provisional application No. 60/392,774, filed on Jun. 28, 2002.

(51) Int. Cl. A61K 9/24

(52) U.S. Cl. 424/473

(57) ABSTRACT

The present invention provides a controlled release oral dosage form for the delivery of a liquid active agent formulation. The dosage form of the present invention includes a reservoir formed of a water impermeable material and filled with a liquid active agent formulation. The dosage form of the present invention also includes an expandable osmotic composition positioned within the reservoir, and, optionally, the expandable osmotic composition is separated from contact with the liquid active agent formulation by a barrier layer that is substantially impermeable to the passage of liquid. To facilitate expulsion of the liquid active agent formulation, the dosage form of the present invention also includes an exit orifice. The material make-up and design of the reservoir included in the dosage form of the present invention allow the manufacture of an oral dosage form capable of more consistently achieving a desired release rate or release rate profile of active agent from a liquid formulation.

This application claims the benefit of U.S. Provisional Application No. 60/392,774, filed Jun. 28, 2002.

### BACKGROUND

1. Field of the Invention

The present invention relates to controlled release dosage forms capable of delivering a liquid active agent formulation. Specifically, the present invention provides a controlled release dosage form that contains a liquid active agent formulation within a reservoir formed of a water impermeable material and is capable of more consistently achieving a targeted release rate or release rate profile.

2. State of the Art

Oral dosage forms providing controlled release of liquid active agent formulations are known in the art. For example, U.S. Pat. No. 6,174,547 (“the '547 Patent”), U.S. Pat. No. 5,830,502 (“the '502 Patent”), U.S. Pat. No. 5,614,578 (“the '578 Patent), International Publication Number WO 95/34285 (“the '285 Publication”), and International Publication Number WO 01/41742 (“the '742 Publication”) teach controlled release dosage forms configured to provide controlled release of liquid active agent formulations. The dosage forms taught in these references include a hard gelatin or injection molded capsule, a liquid active agent formulation contained within the capsule, an expandable osmotic composition positioned within the capsule, a semipermeable membrane formed over the capsule or by the capsule itself, and an exit orifice. As taught in the '285 Publication, the expandable osmotic composition positioned within the capsule may be separated from the liquid active agent formulation by a barrier layer that is substantially impermeable to the passage of liquid. In operation, water from the environment of use is drawn into the expandable osmotic composition through the capsule wall. As water is drawn into the expandable osmotic composition, the composition expands within the capsule and expels the liquid active agent formulation into the environment of use through the exit orifice.

Although the dosage forms taught in the '547 Patent, the '502 Patent, the '578 Patent, '285 Publication, and the '742 Publication are useful to achieve the controlled release of liquid active agent formulations over predetermined periods of time, it has proven surprisingly difficult to consistently achieve targeted release rates using dosage forms manufactured according to such designs. In particular, it has proven difficult to consistently achieve substantially constant release rate profiles using dosage forms manufactured according to the '547 Patent, the '502 Patent, the '578 Patent, '285 Publication, and the '742 Publication. It would be an improvement in the art, therefore, to provide an orally administrable dosage form that not only provides controlled release of a liquid active agent formulation, but also works to reduce or eliminate the undesirable migration of water into the liquid active agent formulation contained within the dosage form. Such a dosage form would work to more consistently achieve targeted release rates, reduce variability in inter-dosage form release rate performance, and increase dosing reliability. Ideally, such a dosage form would work to reduce migration of water into the liquid active agent formulation both before and after administration of the dosage form and could be designed to facilitate delivery of a wide range of active agents from a variety of different liquid formulations. It will be apparent to those skilled in the art that an oral dosage form exhibiting these characteristics would further facilitate the development and commercialization of a dosage forms providing controlled delivery of active agents from liquid active agent formulations.

### SUMMARY OF THE INVENTION

In one aspect, the present invention includes an oral dosage form designed to provide a device that more consistently achieves targeted release rates and release rate profiles of liquid active agent formulations. It has been found that even small fluctuations in the water concentration in a liquid active agent formulation contained within a controlled release dosage form can significantly alter the release rate of active agent achieved by the dosage form. In particular, the concentration of active agent contained within a liquid active agent formulation is diluted if water is drawn into the liquid active agent formulation contained within a dosage form designed for controlled release of the active agent. And, as the concentration of active agent within the liquid active agent formulation is diluted, the amount of active agent delivered from a given amount of liquid active agent formulation is reduced. Therefore, where the design of an oral dosage form allows water to migrate into the liquid active agent formulation before or after administration of the dosage form to an environment of operation, the rate at which the dosage form delivers active agent can be depressed away from a targeted release rate, even where the dosage form delivers the liquid active agent formulation as anticipated. To provide an orally administrable dosage form that more consistently achieves a targeted release rate or release rate profile, the dosage form of the present invention is designed to reduce or prevent the passage of water into the liquid active agent formulation contained therein both before and after administration of the dosage form.

An oral dosage form according to the present invention includes reservoir and a liquid active agent contained within the reservoir. The dosage form of the present invention is configured such that, after administration of the dosage form to a desired subject, the liquid active agent formulation is delivered from the reservoir at a controlled rate over a predetermined period of time. Controlled delivery of the liquid active agent formulation from the reservoir may be achieved using an configuration or combination of elements that are suitable for oral delivery and provide delivery of the liquid active agent at a controlled rate over a predetermined period of time after oral administration. In order to reduce or prevent the passage for water into the liquid active agent formulation, the reservoir included in a dosage form according to the present invention is formed of a material that is impermeable to water. Moreover, the reservoir can be prepared to reduce or minimize the amount of water available to migrate into the liquid active agent formulation from within the material used to form the reservoir itself. By designing the dosage form of the present invention to include a reservoir formed of a water impermeable material, the dosage form of the present invention better facilitates the creation of a dosage form capable of more consistently achieving a targeted active agent release rate profile.

The reservoir included in an oral dosage form according to the present invention can be fabricated using a variety of materials that are impermeable to water or that can be made impermeable to water. In one embodiment, the oral dosage form of the present invention includes a reservoir formed of a single layer of water impermeable material. However, the reservoir included in an oral dosage form of the present invention may also be fabricated using two or more layers of material that, together, are impermeable to water. Therefore, in another embodiment, the dosage form of the present invention includes a reservoir formed of two or more material layers.

In yet another embodiment, the oral dosage form of the present invention includes, a reservoir formed of a water impermeable material, a liquid active agent formulation contained within the reservoir, an expandable osmotic composition positioned at least partially within the reservoir, a semipermeable membrane, an exit orifice that allows the liquid active agent formulation to be expelled from the dosage form. The reservoir included in such an embodiment is configured such that the expandable osmotic composition is not encapsulated by the reservoir forming materials. After administration of the dosage, water passes through the semipermeable membrane into the expandable osmotic composition at a desired rate, and as water enters the expandable osmotic composition the composition expands and acts against the liquid active agent formulation such that the liquid active agent formulation is expelled through the exit orifice at a desired rate over a predetermined period of time.

In another aspect, the present invention provides a method of making a controlled release oral dosage form for the delivery of a liquid active agent formulation. The method of the present invention includes providing a reservoir that is formed of a water impermeable material and is suitable for use in an oral dosage followed by loading the reservoir with a liquid active agent formulation. In one embodiment, the method of the present invention includes providing a reservoir that is formed of a water impermeable material, loading the reservoir with a liquid active agent formulation, positioning an expandable osmotic composition in operative association with the reservoir such that at least a portion of the osmotic composition remains exposed, providing a semipermeable membrane over the exposed portion of the osmotic composition, and forming an exit orifice through which the liquid active agent formulation can be delivered. As will be understood from description that follows, the steps included in the method of the present invention may be embodied by one or more different processes.

### BRIEF DESCRIPTION OF THE DRAWINGS

FIG. 1 provides a schematic illustration of one embodiment of an oral dosage form according to the present invention.

FIG. 2 provides a schematic illustration of a second embodiment of an oral dosage form according to the present invention.

FIG. 3 provides a graph illustrating a release rate profile of acetaminophen achieved using an exemplary oral dosage form according to the present invention.

FIG. 4 provides a graph illustrating a release rate profile of acetaminophen achieved using an oral dosage form lacking a water impermeable reservoir.

FIG. 5 provides a graph illustrating the release rate profile of progesterone achieved using a second exemplary oral dosage form according to the present invention.

FIG. 6 provides a graph illustrating the release rate profile of progesterone achieved by a third exemplary oral dosage form according to the present invention.

### DETAILED DESCRIPTION OF THE INVENTION

The present invention includes an oral dosage form that provides controlled release of liquid active agent formulations. The dosage form of the present invention includes a reservoir formed of a water impermeable material and a liquid active agent formulation contained within the reservoir. The material used to create a reservoir included in a dosage form of the present invention need not be perfectly impermeable to the passage of water. As it is used herein, the term “impermeable” refers to reservoir formed of a material that exhibits a water flux of less than about 10⁻⁴ (mil.cm/atm.hr). The water impermeable nature of the material used to create the reservoir included in a dosage form according to the present invention serves to reduce or prevent migration of water from an external environment, through the reservoir, and into the liquid active agent formulation. The dosage form of the present invention is configured such that, after administration of the dosage form to a desired subject, the liquid active agent formulation is delivered from the reservoir at a controlled rate over a pre-determined period of time. Controlled delivery of the liquid active agent formulation from the reservoir may be achieved using any configuration or combination of elements that are suitable for oral delivery and provide delivery of the liquid active agent at a controlled rate over a predetermined period of time from a reservoir according to the present invention.

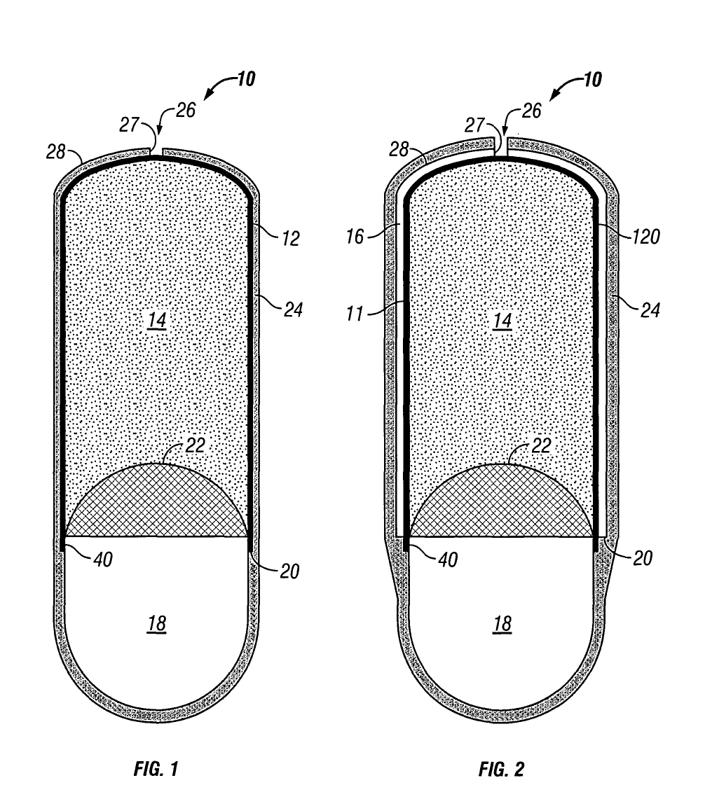

One embodiment of a dosage form 10 according to the present invention is illustrated in FIG. 1. In such an embodiment, the dosage form 10 includes a reservoir 12 formed of a water impermeable material, a liquid active agent formulation 14 contained within the reservoir 12, an expandable osmotic composition 18, a semipermeable membrane 24, and an exit orifice 26. The expandable osmotic composition 18 is positioned within the reservoir 12 such that a portion of the expandable osmotic composition 18 remains exposed, and if desired, the expandable osmotic composition 18 may include a barrier layer 22 that works to separate the expandable portion 19 of the expandable osmotic composition 18 from the liquid active agent formulation 14. Where included, a barrier layer 22 works to prevent mixing of the liquid active agent formulation 14 with the expandable osmotic composition 18 and serves to ensure more complete delivery of the liquid active agent formulation 14 as the dosage form 10 operates. The semipermeable membrane 24 is provided over at least the portion of the expandable osmotic composition 18 that remains exposed after positioning the expandable osmotic composition 18 within the reservoir 12. To facilitate expulsion of the liquid active agent formulation 14, a dosage form 10 of the present invention also includes an exit orifice 26, which is preferably formed in an area near a second end 28 of the reservoir 12. When placed in an environment of operation, the expandable osmotic composition 18 absorbs water at a desired rate through the semipermeable membrane 24. As it absorbs water, the expandable osmotic composition 18 expands within the reservoir 12, causing the expulsion of the liquid active agent formulation 14 from the dosage form 10 through the exit orifice 26.

The reservoir 12 included in an oral dosage form 10 of the present invention is formed to contain a desired amount of liquid active agent formulation and may be formed as desired to accommodate one or more components of a controlled release dosage form 10 of the present invention. For example, where the dosage form is manufactured according to the embodiment illustrated in FIG. 1, the reservoir 12 can be formed with a first end 20 that includes an opening 40 that is sized and shaped to accommodate an expandable osmotic composition 18. Moreover, though the reservoir 12 of an oral dosage form 10 of the present invention may be formed in a generally oblong shape, the dosage form 10 according to the present invention is not so limited and may be manufactured to include a reservoir 12 that is sized and shaped as desired to suit a particular dosage form or drug delivery application.

In the embodiment illustrated in FIG. 1, the reservoir 12 does not completely enclose the expandable osmotic composition 18. In this manner, at least a portion of the expandable osmotic composition 18 remains accessible to water from an outside environment such that the expandable osmotic composition 18 can function to provide controlled release of the liquid active agent formulation 14. Designing the dosage form 10 of the present invention such that the reservoir 12 does not completely enclose the expandable osmotic composition 18 also works to improve -the long-term structural stability of the dosage form 10. In particular, it has been found that the high level of osmotic activity of osmotic compositions included in previous dosage forms designed for the controlled release of liquid formulations can dehydrate the enclosing capsule or reservoir forming materials to such a degree that the material becomes brittle, cracks or is otherwise structurally compromised. The design of the oral dosage form illustrated in FIG. 1, as well as that of the oral dosage form illustrated in FIG. 2, allows contact between the reservoir forming material and the expandable osmotic composition to be minimized, and thereby serves to improve the structural stability of the dosage form 10 over time.

The reservoir 12 included in an oral dosage form 10 of the present invention may be formed of a variety of materials. Any material that is impermeable to water or can be made impermeable to water, is compatible with the desired liquid active agent formulation, is capable of being formed into a desired shape and size, is suitable for use in an oral dosage form, and is capable of withstanding the anticipated storage and operational conditions may be used to provide the reservoir 12 included in a dosage form 10 according to the present invention. The reservoir 12 may be fabricated of a single material or a combination of materials, and where the reservoir 12 includes a combination of materials, the reservoir 12 may be formed of a homogenous or heterogeneous mixture of materials.

In the embodiment illustrated in FIG. 1, an oral dosage form 10 of the present invention includes a reservoir 12 formed in a single layer by a material that is impermeable to the passage of water. Materials suitable for forming such a reservoir include, but are not limited to, water impermeable polymer materials. Where a single layer of water impermeable polymer material is used to form the reservoir 12 included in a dosage form 10 of the present invention, the polymer is preferably a synthetic resin or a combination of synthetic resins. Examples of synthetic resins that may be used to form the reservoir 12 included in a dosage form 10 of the present invention include, for example, linear polycondensation resins, condensation polymerized resins, addition polymerized resins, resins of phthalic anhydrides, polyvinyl resins such as polyethylene, polypropylene and their copolymers, polymer resins of methacrylic acid esters and acrylic acid esters, polycaprolactone, and copolymers of polycaprolactone with dilactide, diglycolide, valerolactone or decalactone. Different impermeable polymer materials and different combinations of impermeable polymer materials may be chosen to provide a reservoir 12 providing desired permeability, compatibility, and stability characteristics.

Where the reservoir 12 of an oral dosage form 10 of the present invention is formed by a single layer of material that is impermeable to the passage of water, the reservoir 12 may be formed using known manufacturing techniques. In one embodiment, the reservoir 12 is formed by coating a mold with a reservoir forming material, such as by dipping the mold into a bath containing the reservoir forming material, cooling the coated mold, drying the mold in a current of air, stripping the lamina of reservoir forming material from the mold to provide a lamina member having an internal lumen, and trimming the lamina member to provide finished reservoir 12. In another embodiment, the reservoir 12 may be formed using an injection molding technology. Injection molding technologies suitable for forming the reservoir 12 included in the dosage form 10 of the present invention are described in U.S. Pat. No. 6,174,547 and U.S. Pat. No. 5,614,578, the contents of both of which are incorporated herein in their entirety by this reference.

In an alternative embodiment, the dosage form 10 of the present invention may include a reservoir 120 formed of two or more layers of different materials. For example, as is illustrated in FIG. 2, a multilayer reservoir 120 of a dosage form of the present invention can be fabricated by coating a water permeable material 11 with a water impermeable subcoat 16. The water permeable material 11 may be formed of a substance that is hydrophilic or otherwise permeable to the passage of water. Such hydrophilic materials include those hydrophilic materials typically used for the formation of capsules for oral delivery of liquid formulations, such as known gelatin and hydrophilic polymer materials. The water permeable material 11 included in a multilayer reservoir 120 of the present invention may also be formed of a combination of water permeable and water impermeable materials, such as the combinations of materials disclosed in U.S. Pat. No. 6,174,547 and U.S. Pat. No. 5,614,578.

Where a multilayer reservoir 120 of a dosage form 10 of the present invention includes a water permeable material 11, however, it is presently preferred that the water permeable material 11 be formed of a hydrophilic polymer material, not a gelatin. The structural stability of gelatin materials, such as the gelatin materials typically used to create capsules for the delivery of liquid formulations, is sensitive to changes in hydration. In particular, it has been found that gelatin materials become brittle and may crack if moisture content drops below about 8%. However, if the moisture content of typical gelatin materials exceeds about 13%, the material can become too soft and tacky for further processing steps, such the process steps necessary to coat the gelatin material with a desired subcoat. Such sensitivity to moisture content is problematic because the liquid active agent formulation 14 and the expandable osmotic composition 18 can exhibit relatively high osmotic activity, which can cause water to migrate out of a gelatin material to such a degree that the material becomes brittle, cracks, or is rendered structurally unsuitable. Therefore, even though gelatin materials may be used to provide a water permeable material 11 in a multilayer reservoir 120 of an oral dosage form 10 of the present invention, such materials are not presently preferred, particularly where liquid active agent formulation 14 included in the dosage form exhibits a relatively high osmotic activity and it is desired that the dosage form have an extended shelf life.

Hydrophilic polymer materials, including cellulosic materials, provide preferred water permeable materials that may be used to form a multilayer reservoir 120 useful in an oral dosage form 10 of the present invention. Relative to the gelatin materials that are typically used in dosage form fabrication, water-soluble polymer materials are less susceptible to moisture loss and are less sensitive to changes in moisture content. As a result, a multilayer reservoir 12 formed using a hydrophilic polymer material is better able to retain its structural integrity upon exposure to the liquid active agent formulation 14 and the expandable osmotic composition 18 included in the dosage form 10 of the present invention. Moreover, because hydrophilic polymer materials can be manufactured with relatively lower moisture content, a multilayer reservoir 120 manufactured using hydrophilic polymer materials can be made such that less water is available to be drawn into the liquid active agent formulation 14 from within the materials forming the multilayer reservoir 120 itself.

Hydrophilic polymer materials that may be used to as the water permeable material 11 included in a multilayer reservoir 120 include, but are not limited to, polysaccharide materials, such as hydroxypropylmethyl cellulose (HPMC), methylcellulose, hydroxyethyl cellulose (HEC), hydroxypropyl cellulose (HPC), poly(vinylalcohol-co-ethylene glycol) and other water soluble polymers. Though the water permeable material 11 included in a multilayer reservoir 120 of a dosage form 10 of the present invention may be manufactured using a single polymer material, the water permeable material 11 may also be formed using a mixture of more than one polymer. Presently, because HPMC capsules for oral delivery of liquid active agent formulations are commercially available and capsule bodies formed of HPMC provide reservoirs exhibiting suitable performance characteristics, the water permeable material 11 included in a multilayer reservoir 120 of a dosage form 10 of the present invention is preferably formed using an HPMC material.

Whether the water permeable material 11 included in a multilayer reservoir 120 included in a dosage form 10 of the present invention is formed using a gelatin material or a polymer material, the water permeable material 11 may be formed to provide a multilayer reservoir 120 of desired shape and capacity using known manufacturing techniques. In particular, the molding and coating techniques described in relation to the formation of a reservoir 12 from a single layer of water impermeable material may be applied to form the water permeable material 11 included in a multilayer reservoir 120. Therefore, in one embodiment, the water permeable material 11 included in a multilayer reservoir 120 is formed by coating a mold, such as by dipping the mold into a bath containing the water permeable material, cooling the coated mold, drying the coated mold in a current of air, stripping the lamina of water permeable material from the mold to provide a lamina member having an internal lumen, and trimming the lamina member to provide a pre-formed water permeable material 11 that will provide a multilayer reservoir of a desired shape and capacity once coated with a water impermeable subcoat 16. In another embodiment, the water permeable material 11 included in a multilayer reservoir 120 may be formed using an injection molding technology, such as the injection molding technology described in U.S. Pat. No. 6,174,547 and U.S. Pat. No. 5,614,578.

A water impermeable subcoat 16 included in a multilayer reservoir 120 of a dosage form according to the present invention may be formed using any suitable water impermeable material that can be coated on or otherwise provided over the water permeable material 11. However, latex materials, such as Surelease® latex materials, which are available from Colorcon, Inc., Kollicoat® SR latex materials, which are available from BASF, Eudragit® SR, and other polymethylacrylate latex materials, are presently preferred for forming a water impermeable subcoat 16. A water impermeable subcoat 16 may be provided over the water permeable material 11 included in a multilayer reservoir 120 of an oral dosage form according to the present invention using any suitable coating or lamination technique. For example, a water impermeable subcoat 16 may be provided over a water permeable material 11 using a dip coating process.

Alternatively, a water impermeable subcoat 16 may be formed over a water permeable material 11 using a spray coating process to provide a multilayer reservoir 120. Where a spray coating process is used, however, the water permeable material 11 is preferably pre-formed to provide a reservoir of desired shape and capacity before the water impermeable subcoat 16 is coated over the water impermeable material 11. In one embodiment, the pre-formed water permeable material 11 to be used in a multilayer reservoir 120 of a dosage form 10 according to the present invention includes an opening 40 provided for positioning an expandable osmotic composition 18 within the multilayer reservoir 120. Preferably, where the pre-formed water permeable material 11 includes an opening 40 for the expandable osmotic composition 18, a removable cap is positioned over the opening 40 before spray coating of the pre-formed water permeable material 11 is conducted. Providing the removable cap prior to the spray coating process prevents unwanted coating of the interior surface of the pre-formed water permeable material 11 with the material forming the water impermeable subcoat 16. Once the spray coating process is complete, however, the cap should be readily removable to allow further processing of the completed multilayer reservoir 120.

Therefore, a spray coating process suitable for coating a capped, pre-formed water permeable material 11 with a water impermeable subcoat 16 to form a multilayer reservoir 120 useful in an oral dosage form 10 of the present invention should be tailored to provide a water impermeable subcoat 16 that is sufficiently robust to allow further processing, while still permitting easy removal of the cap from the completed multilayer reservoir 120. In one embodiment, such a spray coating process is defined by process parameters that provide a water impermeable subcoat 16 that is generally uniform, but is discontinuous at the seam created where the cap overlaps the pre-formed water permeable material 11. Such a subcoat allows the cap to be readily removed from the pre-formed water permeable material 11 without disturbing the water impermeable subcoat 16.

Where a multilayer reservoir 120 according to the present invention includes a water impermeable subcoat 16 formed of a latex material that is spray coated over a pre-formed hydrophilic polymer material, a dry spray coating process is typically used to provide the water impermeable subcoat 16. Dry spray coating processes will generally provide a uniform impermeable subcoat 16 of latex material over a capped, pre-formed hydrophilic polymer material, except at the seam created by the cap, where the coating of latex material will typically be discontinuous. In particular, where a multilayer reservoir 120 is formed using a hydrophilic polymer material spray coated with a water impermeable subcoat of Surelease® or Kollicoat® SR 30D latex material, process parameters providing a suitable multilayer reservoir 120 are described herein in Example 1 through Example 3.

An expandable osmotic composition 18 included in an oral dosage form 10 of the present invention is formulated such that, as it absorbs water from the environment of operation through the semipermeable membrane 24, the expandable osmotic composition 18 expands and exerts a force against the liquid active agent formulation 14 that is sufficient to cause expulsion of the liquid active agent formulation 14 at a desired rate through the exit orifice 26 included in the dosage form. Any composition that exhibits such performance characteristics, is pharmaceutically acceptable, and is compatible with the other components of the dosage form of the present invention may be used to form the expandable osmotic composition 18. In a preferred embodiment, however, the expandable osmotic composition 18 includes a hydrophilic polymer capable of swelling or expanding upon interaction with water or aqueous biological fluids.

An expandable osmotic composition 18 used in a dosage form according to the present invention may further include an osmagent to increase the osmotic pressure exerted by the expandable osmotic composition 18, a suspending agent to provide stability and homogeneity to the expandable osmotic composition 18, a tableting lubricant, an antioxidant, or a non-toxic colorant or dye. As seen in FIG. 1 and FIG. 2, an expandable osmotic composition 18 included in a dosage form 10 of the present invention is preferably provided in a tableted form to ease positioning within an opening 40 formed in the reservoir 12, 120. Materials and methods for forming an expandable osmotic composition 18 suitable for use in an oral dosage form 10 of the present invention are known in the art and are taught, for example, in U.S. Pat. Nos. 6,174,547 and 6,245,357 and in U.S. patent applications Ser. Nos. 08/075,084, 09/733,847, 60/343,001, and 60/343,005, the contents of each of which are herein in their entirety by reference.

As can also be appreciated by reference to FIG. 1 and FIG. 2, an expandable osmotic composition 18 used in a dosage form according to the present invention is preferably tableted in a bi-layer tablet 30 including a barrier layer 22. The barrier layer 22 works to minimize or prevent the mixing of the liquid active agent formulation 14 with the expandable osmotic composition 18 before and during operation of an oral dosage form 10 of the present invention. By minimizing or preventing mixing of the liquid active agent formulation 14 with the expandable osmotic composition 18, the barrier layer 22 serves to reduce the amount of residual active agent remaining within the dosage form 10 after the expandable osmotic composition 18 has ceased expansion or has filled the interior of the dosage form 10. The barrier layer 22 also serves to increase the uniformity with which the driving power of the expandable osmotic composition 18 is transferred to the liquid active agent formulation 14. Where included, the barrier layer 22 is made of a substantially fluid impermeable composition, such as a polymeric composition, a high density polyethylene, a wax, a rubber, a styrene butadiene, a calcium phosphate, a polysilicone, a nylon, Teflon®, a polystyrene, a polytetrafluoroethylene, halogenated polymers, a blend of a microcrystalline, high acetyl cellulose, or a high molecular weight fluid impermeable polymer. Materials and methods suitable for creating a bi-layer tablet 30 including an expandable osmotic composition 18 and a barrier layer 22 are taught, for example, in U.S. patent applications Ser. Nos. 08/075,084, 60/343,001, and 60/343,005, which have already been incorporated herein by reference.

Once tableted, an expandable osmotic composition 18 can be positioned within a reservoir 12, 120 included in a dosage form 10 of the present invention using any suitable apparatus or process. For example, an assembling apparatus, such as an inserter providing insertion depth control or insertion force control can be used to position the tableted expandable osmotic composition 18 within an opening 40 formed within a reservoir 12, 120 included in a dosage form 10 of the present invention.

Where the dosage form of the present invention includes an expandable osmotic composition 18 and a multilayer reservoir 120 including a water permeable material 11 coated with a water impermeable subcoat 16, the expandable osmotic composition 18 is preferably positioned within the reservoir 120 after formation of the water impermeable subcoat 16 in order to ease coating of the water impermeable subcoat 16 over the water permeable material 11. Moreover, where the dosage form of the present invention includes an expandable osmotic composition 18 and a multilayer reservoir 120 or a reservoir 12 formed of a single layer of material, creation of an exit orifice 26 may be simplified by positioning the tableted expandable osmotic composition 18 within the reservoir 12, 120 after the reservoir 12 has been filled with a desired amount of liquid active agent formulation 14. Nevertheless, a tableted expandable osmotic composition 18 may be positioned within the reservoir 12, 120 of an oral dosage form 10 of the present invention either before or after the reservoir 12, 120 is loaded with the liquid active agent formulation 14.

Where a tableted expandable osmotic composition 18 is positioned within the reservoir 12, 120 of an oral dosage form of the present invention before the reservoir 12, 120 is loaded with a liquid active agent formulation 14, an inserter providing insertion depth control is preferably used to position the tableted expandable osmotic composition 18 within the reservoir 12. However, an inserter providing insertion force control is preferably used to position a tableted expandable osmotic composition 18 within a reservoir 12, 120 that has been pre-loaded with a liquid active agent formulation 14.

A semipermeable membrane 24 included on an oral dosage form 10 of the present invention is permeable to the passage of water but is substantially impermeable to the passage of the active agent included in the liquid active agent formulation 14. A semipermeable membrane 24 is non-toxic to the intended subject and maintains its physical and chemical integrity during the operation of the dosage form 10. Further, adjusting the thickness or chemical make-up of the semipermeable membrane 24 can control the rate at which an expandable osmotic composition 18 of included in the dosage form 10 of the present invention expands. Therefore, a semipermeable membrane 24 included in an oral dosage form 10 of the present invention may be used to control the release rate or release rate profile achieved by the dosage form 10.

A semipermeable membrane 24 for use in a dosage form 10 of the present invention may be formed using any material that is permeable to water, is substantially impermeable to the active agent, is pharmaceutically acceptable, and is compatible with the other components of the dosage form of the present invention. Generally, a semipermeable membrane 24 will be formed using materials that include semipermeable polymers, semipermeable homopolymers, semipermeable copolymers, and semipermeable terpolymers. Semipermeable polymers are known in the art, as exemplified by U.S. Pat. No. 4,077,407, which is incorporated herein by this reference, and they can be made by procedures described in Encyclopedia of Polymer Science and Technology, Vol. 3, pages 325 to 354, 1964, published by Interscience Publishers, Inc., New York. A semipermeable membrane 24 included in the dosage form 10 of the present invention may also include a plasticizer to impart flexibility and elongation properties to the semipermeable membrane 24 or a flux regulating agent, such as a flux enhancing or a flux reducing agent, to assist in regulating the fluid permeability or flux through the semipermeable membrane 24.

A semipermeable membrane 24 included in a dosage form 10 according to the present invention is provided over at least the portion of the expandable osmotic composition 18 that is not enclosed within the reservoir 12, 120. However, as is shown in FIG. 1 and FIG. 2, a semipermeable membrane 24 included in a dosage form 10 of the present invention may also be provided over both the reservoir 12, 120 and any exposed portion of the expandable osmotic composition 18. Methods for providing a semipermeable membrane 24 suitable for use in a dosage form according to the present invention are known in the art and include any suitable coating technique, such as a suitable dip coating or spray coating process. Additional references describing materials and methods suitable for fabricating semipermeable membranes suitable for use in an oral dosage form 10 of the present invention include, U.S. Pat. Nos. 6,174,547 and 6,245,357 and U.S. patent applications Ser. Nos. 08/075,084, 09/733,847, 60/343,001, and 60/343,005, the contents which are incorporated in their entirety herein by reference.

The dosage form 10 of the present invention may be provided with any desired liquid active agent formulation 14. As it used herein, the expression “active agent” encompasses any drug, therapeutic compound, or composition that can be delivered to provide a benefit to an intended subject. The expression “liquid active agent formulation” is used herein to indicate a formulation that contains an active agent and is able to flow from the dosage form of the present invention into the environment of use. A liquid active agent formulation 14 suitable for use in the dosage form 10 of the present invention may be neat liquid active agent or a solution, suspension, slurry, emulsion, self-emulsifying composition, liposomal solution, or other flowable formulation in which the active agent is present. The liquid active agent formulation 14 may be a solid, or not flowable, at temperatures lower than the temperature of the operational environment, such as the body temperature of an intended animal or human subject, but such a formulation should become flowable at least after introduction of the dosage form into the operational environment. A binder, antioxidant, pharmaceutically acceptable carrier, permeation enhancer, or the like may accompany the active agent in the liquid active agent formulation 14, and the liquid active agent formulation 14 may include a surfactant of mixture of surfactants. U.S. Pat. Nos. 6,174,547 and 6,245,357 and U.S. patent applications Ser. No. 08/075,084, 09/733,847, 60/343,001, and 60/343,005, which are incorporated herein in their entirety by reference, detail exemplary drugs, carriers, and other constituents that may be used to form a liquid active agent formulation suitable for use in the dosage form of the present invention.

An exit orifice 26 included in an oral dosage form 10 of the present invention may be embodied by one of various different structures suitable for allowing the release of the liquid active agent formulation 14. For example, as is shown in the FIG. 1 and FIG. 2, the exit orifice 26 included in a dosage form according to the present invention may simply include an aperture 27 formed through a semipermeable membrane 24, or the exit orifice may include an aperture 27 formed through a semipermeable membrane 24 and a water impermeable subcoat 16 of dosage form 10 that includes a multilayer reservoir 120. An exit orifice 26 formed of an aperture 17 as illustrated in FIG. 1 and FIG. 2 may be formed by any suitable means, such as by suitable mechanical or laser drilling technologies.

Though the aperture 27 illustrated in FIG. 1 and FIG. 2 does not pass entirely through the reservoirs 12, 120 included in the dosage forms 10 illustrated in the figures, the aperture 27 allows the formation of an exit orifice as the dosage form is placed within or begins to operate within an environment of operation. In particular, where a dosage form 10 of the present invention includes a reservoir 12 formed of a single layer of water impermeable material, the aperture 27 formed in the semipermeable membrane 24 creates a breaking point where the material forming the reservoir 12 is compromised as the expandable osmotic composition 18 included in the dosage form 10 begins to function and pressure within the reservoir 12 builds. Alternatively, where a dosage form 10 of the present invention includes a multilayer reservoir 120 and the aperture 27 exposes the water permeable material 11 included in the multilayer reservoir 120, the water present in the environment of operation can work to weaken or dissolve the exposed portion reservoir 120, allowing the liquid active agent formulation 14 contained within the reservoir 12 to be expelled as the expandable osmotic composition 18 expands and acts against the liquid active agent formulation 14.

Nevertheless, the dosage form 10 of the present invention is not limited to an exit orifice 26 formed of an aperture 27 as illustrated in FIG. 1 and FIG. 2. Where desired, the exit orifice may include an aperture that passes completely through the semipermeable membrane and the reservoir. Again, mechanical or laser drilling technologies may be used to create such an exit orifice. However, where the exit orifice provided in the dosage form of the present invention is formed through the reservoir, a closure sealing the exit orifice must generally be provided. Any one of several means may be employed to provide such a closure. For instance, the closure may include a layer of material that covers the exit orifice and is arranged over a portion the outer surface of the dosage form, or the closure may include a stopper, such as a bung, cork, or impermeable plug, or an erodible element, such as a gelatin plug or a pressed glucose plug, formed or positioned within the exit orifice. Regardless of its specific form, the closure will comprise a material impermeable to the passage of the liquid active agent formulation, at least until after administration of the dosage form. Suitable closure materials not already mentioned include high-density polyolefin, aluminized polyethylene, rubber, silicon, nylon, synthetic fluorine Teflon®, chlorinated hydrocarbon polyolefins, and fluorinated vinyl polymers.

An exit orifice included in a dosage form of the present invention may also include more than a simple aperture, where desired, the exit orifice may include, for example, a porous element, porous overlay, porous insert, hollow fiber, capillary tube, microporous insert, or microporous overlay. Moreover, regardless of the particular structure providing the exit orifice, a controlled release dosage form of the present invention can be manufactured with two or more exit orifices for delivering the active agent formulation during operation. Descriptions of exit orifices suitable for use in controlled release dosage forms are disclosed, for example, in those patents and patent applications already incorporated herein by reference, as well as in U.S. Pat. Nos. 3,845,770, 3,916,899, and 4,200,098, the contents of which are herein incorporated in their entirety by reference.

Though an exit orifice 26 formed of an aperture 27 as illustrated in FIG. 1 and FIG. 2 is only one of various different exit orifices that may be provided in a dosage form 10 of the present invention, exit orifices 26 that are formed as shown in FIG. 1 and FIG. 2 are advantageous, as they do not require complete penetration of the reservoir 12, 120 before the dosage form 10 is administered. Such a design works to reduce the possibility that the liquid active agent formulation 14 may leak from the dosage form 10 before the dosage form 10 is administered. Moreover, the aperture 27 included in the exit orifices 26 shown in FIG. 1 and FIG. 2 is simply formed using known mechanical or laser drilling techniques.

### EXAMPLE 1

Dosage forms according to the present invention were manufactured. The exemplary dosage forms were manufactured according to the design illustrated in FIG. 2. That is, the exemplary dosage forms included a multilayer reservoir, with the reservoir being formed of a water-soluble polymer coated within a water impermeable subcoat. The reservoir of the exemplary dosage forms was filled with a liquid active agent formulation, and the exemplary dosage forms were provided with an expandable osmotic composition that was tableted into a bi-layer tablet including the expandable osmotic composition and a barrier layer. The exemplary dosage forms were coated with a semipermeable membrane and provided with an exit orifice formed by an aperture that initially extended through both the semipermeable membrane and the water impermeable subcoat. The release rate performance of the exemplary dosage forms was evaluated and compared with the release rate performance achieved by dosage forms that did not incorporate a water impermeable subcoat.

The bilayer tablet including the expandable osmotic composition and the barrier layer was manufactured using standard granulation and tableting techniques. The expandable osmotic composition was by first sizing and screening NaCl using a 21-mesh screen and a Quardo Mill set at the maximum speed. Once the NaCl was sized and screened the following dry ingredients were added to and blended in a granulator bowl: 73.70 wt % polyethylene oxide 303, 20.00 wt % NaCl, and 1.00 wt % iron oxide green. In a separate container, a granulating solution was prepared by dissolving 5.00 wt % PVP K29 in purified water. The blended dry ingredients were fluidized in a Glatt Fluid Bed Granulator, and the granulating solution was sprayed onto the fluidized dry ingredients until all of the solution was applied and a granular composition was formed. 0.25 wt % stearic acid and 0.05 wt % BHT were blended with the granular composition to provide an expandable osmotic composition ready for tableting. Two hundred and fifty milligrams of the granular expandable osmotic composition were added to 0.71 cm punch (modified ball lower punch and modified upper punch) and tamped to provide the tableted expandable osmotic composition portion of the bilayer tablet.

The barrier layer composition was also granulated using a Glatt FBG. To prepare the barrier layer composition Microfine wax and Kolidone SR were blended in a granulator bowl. In a separate container, a granulating solution was prepared by dissolving PVP 29 into purified water. The blended Microfine wax and Kolidone SR were fluidized in the Glatt FBG and the granulating solution was sprayed onto the fluidized constituents until all of the solution was applied and a granular composition was formed. The granulated barrier layer composition included 45.87 wt % Microfine wax, 45.87 wt % Kolidone SR, and 8.26 wt % PVP K29. After the 250 mg of the expandable osmotic composition had been added to the 0.71 cm punch and tamped, 100 mg of the granulated barrier layer composition was added to the punch. The tamped expandable osmotic composition and the barrier layer composition were then compressed using a Korsch press to form a bi-layer tablet including both the expandable osmotic composition and the barrier layer.

The reservoir included in the exemplary dosage forms was provided using clear, size-0 HPMC Vcaps™ capsules supplied by Capsugel®, with the water permeable material of the reservoirs being formed by the capsule bodies of the Vcaps™ capsules. Before the caps of the Vcaps™ capsules were removed from the capsule bodies, the capsules were coated with a water impermeable subcoat formed of Kollicoat® SR latex. To coat the capsules, a coating suspension of 97 wt % Kollicoat® SR and 3 wt % propylene glycol was prepared. The capsules were then coated by applying the prepared coating composition in a 24″ Hi-coater under the process conditions detailed in Table 1. Under these process conditions, the capsules were coated with a Kollicoat® SR subcoat that was continuous over the capsule bodies but discontinuous at the seam between the capsule caps and the capsule bodies. The caps were, therefore, easily separated from the capsule bodies without disturbing the newly applied Kollicoat® SR subcoat, thereby providing completed multilayer reservoirs.

After completion, the multilayer reservoirs were then loaded with 500 mg of a liquid active agent formulation. The liquid active agent formulation included in the exemplary dosage forms included, by weight, 5% acetaminophen and 95% Cremophor EL. The liquid active agent solution was prepared and loaded using standard manufacturing techniques.

Once the multilayer reservoirs were loaded with the liquid active agent formulation, a bi-layer tablet including the expandable osmotic composition having a barrier layer was positioned within the open end of each of the multilayer reservoirs, creating pre-coating assemblies. The bi-layer tablets were positioned within the filled multilayer reservoirs using an inserter providing insertion force control and the bi-layer tables were oriented within the multilayer reservoirs such that the barrier layer was facing the liquid active agent formulation, thereby isolating the expandable osmotic composition from the liquid active agent formulation. The bi-layer tablets were positioned within the filled multilayer reservoirs using an inserter providing insertion force control.

The exemplary dosage forms were then completed by coating the precoating assemblies (including the multilayer reservoir filled with the liquid active agent formulation and having an expandable osmotic composition positioned therein) with a semipermeable membrane followed by providing the coated assemblies (including the pre-coating assemblies coated with a semipermeable membrane) with an exit orifice. The semipermeable membrane provided on the pre-coating assemblies included 85 wt % cellulose acetate 398-10 and 15 wt % Pluronic F-68. The semipermeable membrane was coated on the pre-coating assemblies using a coating solution formed by dissolving the desired amount of cellulose acetate 398-10 and Pluronic F-68 in acetone to provide a coating solution with a solid content of 4 wt %. The coating solution was then spray coated onto the pre-coating assemblies in a 12″ Freud Hi-coater until each of the pre-coating assemblies were coated with about 76 mg of the semipermeable membrane composition. Each of the coated assemblies was then provided with an exit orifice including an aperture having a 20 mil (0.5 mm) diameter formed through the semipermeable membrane and the water impermeable subcoat included on the multilayer reservoirs. The exit orifices were created using a mechanical drill with drilling depth control. The exemplary dosage forms were then dried at 45° C. and 45% relative humidity for one day followed by an additional day of drying at 45° C. and ambient relative humidity.

After drying, the release rate profile of acetaminophen provided by the exemplary dosage forms was measured. Three of the exemplary dosage forms were chosen and the release rate profile provided by the exemplary dosage forms was measured using a USP VII method in simulated intestinal fluid without enzyme (pH 6.8). The release rate profile of acetaminophen achieved by the exemplary dosage forms is illustrated in FIG. 2. As can be appreciated by reference to FIG. 2, the exemplary dosage forms achieved a substantially constant release of acetaminophen over an approximately 16-hour period of time.

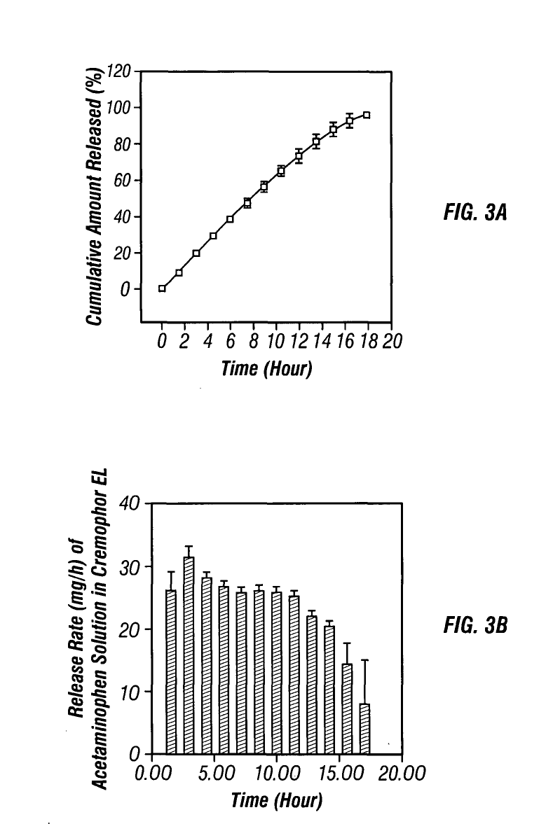

For comparison, the release rate performance achieved by dosage forms that did not include a reservoir formed of a water impermeable material was also evaluated. The reservoirs of the dosage forms used for the comparative release rate evaluation were formed using the capsule bodies of clear, size-0 HPMC Vcaps™ capsules supplied by Capsugel®. However, the capsule bodies forming the reservoirs of the dosage forms used in the comparative release rate evaluation were not coated with a water impermeable subcoat. Except for reservoirs used in the dosage forms, the dosage forms used in the comparative release rate evaluation were manufactured just as the exemplary dosage forms were manufactured. Three dosage forms were selected for comparative purposes, and the release rate of acetaminophen achieved by the three dosage forms lacking a reservoir formed of a water impermeable material was evaluated using a USP VII method conducted in simulated intestinal fluid without enzyme (pH 6.8). FIG. 3 illustrates the results achieved by the three dosage forms lacking a reservoir formed of a water impermeable material. As can be seen in FIG. 3, the release rate of acetaminophen provided by the dosage forms that did not include a reservoir formed by a water impermeable material is noticeably less constant over the time required to release substantially all the acetaminophen from the dosage forms.

### EXAMPLE 2

A second exemplary dosage form according to the present invention was manufactured and evaluated. The second exemplary dosage form was manufactured according to the procedure for manufacturing the exemplary dosage form of Example 1, except that Surelease® was the latex material used to provide a water impermeable subcoat in the multilayer reservoir and the multilayer reservoir of the second exemplary dosage form was filled with 500 mg of a liquid active agent formulation that included, by weight percent, 2% progesterone and 98% Myvacet 9-45. As was done in Example 1, the water impermeable subcoat was coated in a 24″ Hi-coater and the spray coating process was defined by the process parameters detailed in Table 1. These process conditions yielded a water impermeable subcoat formed of Surelease® latex material that was continuous over the reservoir, yet discontinuous at the seam between the reservoir and the cap.

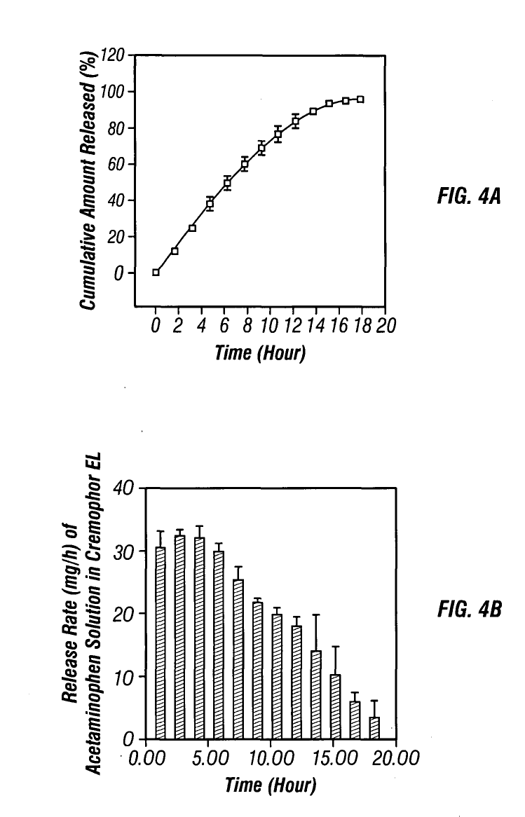

The release rate profile achieved by the second exemplary dosage form was measured. To measure the release rate profile achieved by the second exemplary dosage form, three of the second exemplary dosage forms were evaluated using a USP VII method in simulated intestinal fluid without enzyme (pH 6.8). The release rate profile of progesterone achieved by the second exemplary dosage form is illustrated in FIG. 4.

### EXAMPLE 3

A third exemplary dosage form according to the present invention was manufactured and evaluated. The third exemplary dosage form was manufactured according to the procedure set forth in Example 2, except that the multilayer reservoir of the third exemplary dosage form was filled with 500 mg of a liquid active agent formulation including, by weight percent, 2% progesterone, 49% Myvacet 9-45, and 49% Cremophor EL.

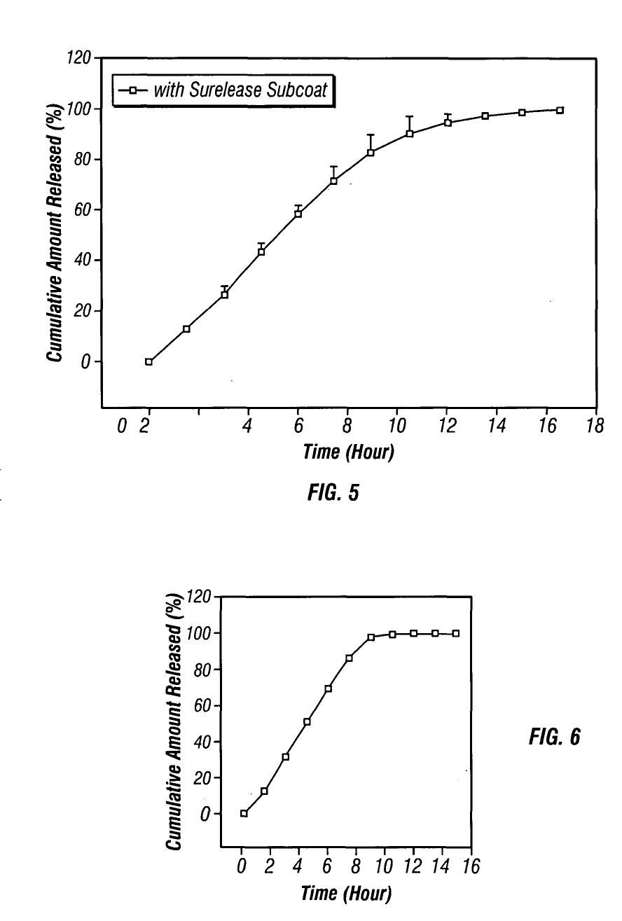

The release rate profile achieved by the third exemplary dosage form was measured. To measure the release rate profile achieved by the third exemplary dosage form, three of the third exemplary dosage forms were evaluated using a USP VII method in simulated intestinal fluid without enzyme (pH 6.8). The release rate profile of progesterone achieved by the third exemplary dosage form is illustrated in FIG. 5.

TABLE 1

| Pan Coater | Target |
|---|---|
| Nozzle Size (mm) | 012 |
| Nozzle Air Cap No. (mm) | 015 |
| Atomization Air Flow Rate (slpm) | 80 |
| Pattern Air Flow Rate (slpm) | 30 |
| Gun Air Flow Rate (slmp) | 110 |
| Pump Speed (mL/min/gun) | 15 |
| Pan Speed (rpm) | 15 |
| Inlet Air Temperature (C.) | Adjust as needed to maintain |
| | exhaust temperature |
| Exhaust Air Temperature (C.) | 28 |
| Gun-To-Bed Distance (inches) | 6 |
| Inlet Air Flow (cfm) | 325 |
| Pan Pressure (inches of water) | −1 |

We claim:

1. An oral dosage form configured to provide controlled release of a liquid active agent formulation, the dosage form comprising:
a reservoir formed of a material that is impermeable to the passage of water, the liquid active agent formulation being contained within the reservoir; and
a delivery means for expelling the liquid active agent formulation from oral dosage form at a controlled rate positioned at least partially within the reservoir.

2. The oral dosage form of claim 1, wherein the reservoir is formed of a single layer of material comprising a water impermeable polymer.

3. The oral dosage form of claim 1, wherein the reservoir is formed of a single layer of material comprising a water impermeable polymer selected a group of water impermeable polymers consisting of linear polycondensation resins, condensation polymerized resins, addition polymerized resins, resins of phthalic anhydrides, polyvinyl resins such as polyethylene, polypropylene and their copolymers, polymer resins of methacrylic acid esters and acrylic acid esters, polycaprolactone, and copolymers of polycaprolactone with dilactide, diglycolide, valerolactone or decalactone.

4. The oral dosage form of claim 1, wherein the reservoir is a multilayer reservoir comprising a water permeable material and a water impermeable subcoat provided over the water permeable material.

5. The oral dosage form of claim 1, wherein the reservoir is a multilayer reservoir comprising a hydrophilic polymer material and a water impermeable subcoat provided over the hydrophilic polymer material.

6. The oral dosage form of claim 1, wherein the reservoir is a multilayer reservoir comprising a hydrophilic polymer material and a water impermeable subcoat provided over the hydrophilic polymer material, wherein the hydrophilic polymer material is selected from a polysaccharide material and a poly(vinylalcohol-co-ethylene glycol).

7. The oral dosage form of clam 6, wherein the polysaccharide material is selected from a group consisting of hydroxypropylmethyl cellulose (HPMC), methylcellulose, hydroxyethyl cellulose (HEC), and hydroxypropyl cellulose (HPC).

8. The oral dosage form of claim 1, wherein the reservoir is a multilayer reservoir comprising a water permeable material and a water impermeable subcoat provided over the water permeable material, the water impermeable subcoat being formed of a water impermeable latex material.

9. The oral dosage form of claim 1, wherein the reservoir is a multilayer reservoir comprising a water permeable material and a water impermeable subcoat provided over the water permeable material, the water impermeable subcoat being formed of a polymethylacrylate latex material.

10. The oral dosage form of claim 1, wherein the reservoir is a multilayer reservoir comprising a gelatin material and a water impermeable subcoat provided over the gelatin material.

11. The oral dosage form of claim 1, wherein the reservoir includes an opening and an expandable osmotic composition positioned within the opening, with the reservoir and the opening being configured such that the expandable osmotic composition is not completely encapsulated by the reservoir.

12. The oral dosage form of claim 1, wherein the delivery means for expelling the liquid active agent formulation from oral dosage form at a controlled rate comprises an expandable osmotic composition positioned within the reservoir such that a portion of the exapandable osmotic composition is not enclosed by the reservoir.

13. An oral dosage form comprising:
a reservoir formed of a water impermeable material, the reservoir comprising an opening;
a liquid active agent formulation contained within the reservoir;
an expandable osmotic composition positioned within the opening of the reservoir such that at least a portion of the expandable osmotic composition is not enclosed by the reservoir;
a semipermeable membrane formed over the portion of the expandable osmotic composition that is not enclosed by the reservoir; and
an exit orifice.

14. The oral dosage form of claim 13, wherein the reservoir is formed of a single layer of water impermeable material.

15. The oral dosage form of claim 13, wherein the reservoir is formed of a single layer of a water impermeable polymer material.

16. The oral dosage form of claim 13, wherein the reservoir is formed of a single layer of water impermeable polymer material selected from a group consisting of linear polycondensation resins, condensation polymerized resins, addition polymerized resins, resins of phthalic anhydrides, polyvinyl resins such as polyethylene, polypropylene and their copolymers, polymer resins of methacrylic acid esters and acrylic acid esters, polycaprolactone, and copolymers of polycaprolactone with dilactide, diglycolide, valerolactone or decalactone.

17. The oral dosage form of claim 13, wherein the reservoir is a multilayer reservoir comprising a water permeable material and a water impermeable subcoat provided over the water permeable material.

18. The oral dosage form of claim 13, wherein the reservoir is a multilayer reservoir comprising a hydrophilic polymer material and a water impermeable subcoat provided over the hydrophilic polymer material.

19. The oral dosage form of claim 13, wherein the reservoir is a multilayer reservoir comprising a hydrophilic polymer material and a water impermeable subcoat provided over the hydrophilic polymer material, wherein the hydrophilic polymer material is selected from a polysaccharide material and a poly(vinylalcohol-co-ethylene glycol).

20. The oral dosage form of clam 13, wherein the polysaccharide material is selected from a group consisting of hydroxypropylmethyl cellulose (HPMC), methylcellulose, hydroxyethyl cellulose (HEC), and hydroxypropyl cellulose (HPC).

21. The oral dosage form of claim 13, wherein the reservoir is a multilayer reservoir comprising a water permeable material and a water impermeable subcoat provided over the water permeable material, the water impermeable subcoat being formed of a water impermeable latex material.

22. The oral dosage form of claim 13, wherein the reservoir is a multilayer reservoir comprising a water permeable material and a water impermeable subcoat provided over the water permeable material, the water impermeable subcoat being formed of a polymethylacrylate latex material.

23. The oral dosage form of claim 13, wherein the reservoir is a multilayer reservoir comprising a gelatin material and a water impermeable subcoat provided over the gelatin material.

24. The oral dosage form of claim 13, wherein the semipermeable membrane extends over an outer surface of the reservoir.

25. A method for making an oral dosage form, the method comprising:
providing a reservoir formed of a water impermeable material and including an opening;
loading the reservoir with a liquid active agent formulation;
positioning an expandable osmotic composition within the opening included in the reservoir such that a portion of the expandable osmotic composition remains exposed;
providing a semipermeable membrane over at least the exposed portion of the expandable osmotic composition; and
creating an exit orifice that allows delivery of the liquid active agent formulation from the dosage form.

26. The method according to claim 25, wherein providing a reservoir includes providing a reservoir formed of a single layer of material.

27. The method according to claim 25, wherein providing a reservoir includes providing a reservoir formed of a water impermeable polymer material.

28. The method according to claim 25, wherein providing a reservoir includes providing a reservoir formed of a water impermeable polymer material selected from linear polycondensation resins, condensation polymerized resins, addition polymerized resins, resins of phthalic anhydrides, polyvinyl resins such as polyethylene, polypropylene and their copolymers, polymer resins of methacrylic acid esters and acrylic acid esters, polycaprolactone, and copolymers of polycaprolactone with dilactide, diglycolide, valerolactone or decalactone.

29. The method according to claim 25, wherein providing a reservoir includes providing a multilayer reservoir comprising a water permeable material and a water impermeable subcoat provided over the water permeable material.

30. The method according to claim 25, wherein providing a reservoir includes providing a multilayer reservoir comprising a hydrophilic polymer material and a water impermeable subcoat provided over the hydrophilic polymer material.

31. The method according to claim 25, wherein providing a reservoir includes providing a multilayer reservoir comprising a hydrophilic polymer material and a water impermeable subcoat provided over the hydrophilic polymer material, wherein the hydrophilic polymer material is selected from a polysaccharide material and a poly(vinylalcohol-co-ethylene glycol).

32. The method according to claim 31, wherein providing a multilayer reservoir comprising a hydrophilic polymer material comprises providing a multilayer reservoir including a hydrophilic polymer material selected from a group consisting of hydroxypropylmethyl cellulose (HPMC), methylcellulose, hydroxyethyl cellulose (HEC), and hydroxypropyl cellulose (HPC).

33. The method according to claim 25, wherein providing a reservoir includes providing a multilayer reservoir comprising a water permeable material and a water impermeable subcoat provided over the water permeable material, the water impermeable subcoat being formed of a water impermeable latex material.

34. The method according to claim 25, wherein providing a reservoir includes providing a multilayer reservoir comprising a water permeable material and a water impermeable subcoat provided over the water permeable material, the water impermeable subcoat being formed of a polymethylacrylate latex material.

35. The method according to claim 25, wherein providing a reservoir includes providing a multilayer reservoir comprising a gelatin material and a water impermeable subcoat provided over the gelatin material.

36. The method according to claim 25, wherein providing a semipermeable membrane comprises providing a semipermeable membrane that extends over an outer surface of the reservoir.
